(19) United States
(12) Patent Application Publication
(10) Pub. No.: US 2024/0302356 A1
(43) Pub. Date: Sep. 12, 2024

(54) METHOD FOR DETERMINING A TOXICITY AND/OR GROWTH PROMOTION EFFECT OF A TREATMENT OR COMPOUND

(71) Applicant: UNIVERSITEIT HASSELT, Hasselt (BE)

(72) Inventors: Ann CUYPERS, Hasselt (BE); Marijke JOZEFCZAK, Hasselt (BE); Sophie HENDRIX, Hasselt (BE)

(21) Appl. No.: 18/263,997

(22) PCT Filed: Feb. 3, 2022
(86) PCT No.: PCT/EP2022/052518

§ 371 Date: Aug. 2, 2023

(30) Foreign Application Priority Data
Feb. 5, 2021 (EP) 21155563.6

(51) Int. Cl. G01N 33/50

(52) U.S. Cl. CPC G01N 33/5097

(57) ABSTRACT

The present invention relates to the field of screening methods. More specifically, the present invention pertains to a method for determining a toxicity and/or growth promotion effect of any one of a treatment or compound, and a computer program comprising instructions to carry out steps of said method.

### FIELD OF THE INVENTION

The present invention relates to the field of screening methods. More specifically, the present invention pertains to a method for determining a toxicity and/or growth promotion effect of any one of a treatment or compound, and a computer program comprising instructions to carry out steps of said method.

### BACKGROUND TO THE INVENTION

Before the development of agriculture, the hunter-gatherer lifestyle supported about 4 million people globally, whereas today modern agriculture feeds more than 7 billion people worldwide. In order to keep up with an increasing overpopulation of planet earth, and its food needs, the role of agriculture is even more critical than before. It has been estimated that the global use of nitrogen (N) and phosphorus (P) fertilizers increased by 7 and 3.5 fold, respectively, in the past 6 decades; and is expected to increase further threefold by 2050, featuring a conversion of agricultural practices more and more towards intensive agricultural practices, wherein higher crop yields are achieved by means of a large use of fertilizers and pesticides necessary to crop growing. This abundant use of fertilizers and pesticides also increases the risk that nutrients and pesticides run-off into surface and leach into groundwater. Therefore, the use of intensive agricultural practices can be at the expense of the environment. Intensive agricultural practices are based on the large application to crops of fertilizers and pesticides, which increases the risk of contamination from nutrients and toxic chemical compounds in groundwater and surface waters, incurring in eutrophication and soil quality degradation. In this context, the screening of both soil quality and the effects of e.g. fertilizers and pesticides have on crops is really important for allowing farmers to grow healthy crops in high yields whilst preserving the soil health.

The problem in the state of the art is that there is no fast and sensitive screening method to measure the quality of soil and the effect of substrate amendments on crops, such, as biochar, toxic substances (metals, organic pollutants, pesticides, . . . ), the excess of nutrients (salts, nitrogen, phosphate, . . . ), growth-promoting substances (hormones, micro-organisms, . . . ), biotic stress (pathogens, . . . ), . . . . Biochar is a carbonized material, rich in carbon, that can be used as an additive in soils or substrates (i.e., growing media such as potting soil) with the aim of increasing its agricultural value in a sustainable and environmentally friendly way as an agro-ecological application. Biochar is created by pyrolysis of organic biomass with a temperature between 400 and 700° C. in the absence of oxygen. Thanks to its stable carbon matrix, biochar has a strong adsorbing capacity for water, gases, nutrients and pollutants (including metals, pesticides, . . . ). The specific characteristics of a biochar type depend on the raw material used as input material as well as on the process parameters during production. Due to the wide variety of biochars, there is a need for a rapid screening method to predict the effects on plant growth.

Olszyk et al., 2018, describe a method to estimate the effect biochar has on different plant species. They evaluate the effect of biochar based on its effect on the germination of seeds of different plants. Olszyk et al., 2018 has several drawbacks, first, the plant germination data alone does not directly give an indication of plant growth, and should be combined with other data to make predictions on the longer term, requiring additional measurements, and secondly, germination and growth tests are not very sensitive as they rely on phenotypical/visual evaluation. These shortcomings make methods to estimate toxicity and/or growth promotion of any one of a treatment or component slow, process intensive and not particularly accurate. Therefore, there is an industry need for a method for determining toxicity and/or growth promotion of any one of a treatment or component, such as a carbonized material, which overcomes the drawbacks of the prior art.

### SUMMARY OF THE INVENTION

In a first aspect, the present invention relates to a method for determining a toxicity and/or growth promotion effect of any one of a treatment or compound, comprising the steps of:
a) providing at least one plant system or parts thereof comprising at least one cell which ploidy is provided to change on exposure to the any one of a treatment or compound;
b) exposing the plant system or parts thereof to the treatment or compound;
c) providing at least a first and/or a second series of one or more ploidy levels of the nuclei in a sample of the exposed plant system or parts thereof, wherein
   the first series is calculated from a basic ploidy level up to, and including, a ploidy level tipping point T corresponding to an arrest/inhibition towards higher ploidy levels for the sample; and
   the second series is calculated from, and excluding, the ploidy level tipping point T to the highest ploidy level observed for the plant system; and
d) determining the toxicity and/or growth promotion effect of the treatment or compound in the plant system based on the first series and/or second series of ploidy levels provided at step c). An advantage of the first aspect of the present invention is that it allows the determination of the effects of a treatment or compound on plant responses in a fast way and independently of treatment or compound's physico-chemical parameters or plant system phenotypical analyses. Further, an advantage of the present embodiment is that the effect a treatment or compound has on a plant system can be determined accurately and with high sensitivity in a reduced amount of time and it is less labour intensive.

In accordance with an embodiment of the present invention, step d) of determining the toxicity and/or growth promotion effect is based on one or more of an endoreplication index (EI) calculated in accordance with formula (I) and/or formula (II)

$${EI}_{DEFENCE} = {\sum }_{y = 0}^{y = a} ( {x}_{y} \times {Z}_{y} ) \qquad ( I )$$

$${EI}_{GROWTH} = {\sum }_{y = a + 1}^{y = k} ( {x}_{y} \times {Z}_{y} ) \qquad ( II )$$

wherein EI_{DEFENCE} is a defence endoreplication index calculated in accordance with the first series provided at step c), and EI_{GROWTH} is a growth endoreplication index calculated in accordance with the second series provided at step c), and wherein
   y is the index of each summation and a non-negative integer referring to the number of cell cycles, for which y $1, or the number of endocycles plus 1, for which y>1,
   x_{y} is an element of the real numbers,
   Z_{y} is the fraction of nuclei in a sample of the exposed plant system or parts thereof of a represented ploidy level P^{y} DNA copies, wherein P is the basic ploidy level of said plant system,
   k is equal to log_{P}(Q), wherein Q is the highest ploidy level of said plant system,
   a is equal to log_{P}(T), wherein T is the ploidy level tipping point of said plant system.

In accordance with a further embodiment of the present invention, the ploidy level tipping point T is defined as the lowest ploidy level for which its relative abundance ratio with respect to its subsequent ploidy level, shows a dose-dependent increase with the treatment or compound within a certain range.

In accordance with a further embodiment of the present invention, at step d) the toxicity effect is determined based on the first series provided at step c) or EI_{DEFENCE}, and/or the growth promotion effect is determined based on the second series provided at step c) or EI_{GROWTH}. In accordance with a further embodiment, the toxicity effect on the plant system is determined by means of EI_{DEFENCE} of the plant system being higher than a EI_{DEFENCE} for a control, and/or the growth promotion effect on the plant system is determined by means of EI_{GROWTH} for the plant system being higher than a EI_{GROWTH} for a control.

In accordance with a further embodiment of the present invention, at step a) the plant system is provided in a growth matrix to which the treatment or compound of step b) is applied.

In accordance with a further embodiment of the present invention, the growth matrix is a liquid medium, soil or substrate for cultivating the plant system.

In accordance with a further embodiment of the present invention, at step a) the plant system is selected from: Arabidopsis thaliana, Dianthus caryophyllus, . . . ; preferably Arabidopsis thaliana. It has been found that plant systems in accordance with the present embodiment provide for satisfactory results and are easy to setup.

In accordance with a further embodiment of the present invention, at step b) the sample of the exposed plant system is a seedling of the plant system.

In accordance with a further embodiment of the present invention, at step d) the toxicity and/or growth promotion effect of the treatment or compound is determined based on data measured by means of flow cytometry.

An advantage of the present embodiment is that the technical implementation is simple and does not influence the results' interpretation, making the results very robust and reliable. Further, in accordance with the present embodiment the screening is very sensitive and objective and it can be performed at very low concentrations of treatments or compounds and low biomass is needed from the plant or plants thereof, which is beneficial to certain applications.

In accordance with a further embodiment of the present invention, at step a) the plant system is provided in a microtiter plate. An advantage of the present embodiment is that only a limited space is required to test or screen multiple conditions.

In accordance with a further embodiment of the present invention, at step b) the compound is a carbonized material, such as conventional pyrolysis (CPS) carbonized material, conventional pyrolysis including a washing step and degassing after grinding (CPS-W/OG) obtained carbonized material or microwave pyrolysis (MWP) obtained carbonized material.

In a second aspect, the present invention relates a computer program comprising instructions which, when the program is executed by a computer, cause the computer to carry out the steps c) and/or d) of the method according to the present invention.

### BRIEF DESCRIPTION OF THE DRAWINGS

With specific reference to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. No attempt is made to show structural details of the invention in more detail than necessary for its fundamental understanding. The description taken with the drawings making apparent to those skilled in the art how the several forms of the invention may be embodied in practice.

FIG. 1, also abbreviated as FIG. 1, illustrates photos of 7-days-old A. thaliana, grown in liquid growth medium in a 96-well microtiter plate without (left) and with (right) addition of biochar (FIG. 1A) and photos of 9-days-old D. caryophyllus, grown in liquid growth medium in a 48-well microtiter plate without (left) and with (right) addition of 30 μM cadmium (Cd) (FIG. 1B). FIG. 2, also abbreviated as FIG. 2, illustrates polyaromatic hydrocarbons (PAHs) concentrations in 3 different biochar types. FIG. 3, also abbreviated as FIG. 3, illustrates the average number of nuclei per microliter of carrier fluid of analysed plant samples from a plant system exposed to different biochar types at different percentages (m %). FIG. 4, also abbreviated as FIG. 4, illustrates the endoreplication index for growth/homeostasis (EI_{GROWTH}, left) and for stress/defence (EI_{DEFENCE}, right) of the analysed plant samples subjected to the same biochar percentages (m %) in FIG. 3. FIG. 5, also abbreviated as FIG. 5, illustrates the average number of nuclei per microliter of carrier fluid of analysed plant samples from a plant system exposed to different biochar types at different percentages (m %) with or without the addition of different cadmium (Cd) concentrations in the range of 0-50 μM Cd. FIG. 6, also abbreviated as FIG. 6, illustrates the endoreplication index for growth/homeostasis (EI_{GROWTH}, top) and for stress/defence (EI_{DEFENCE}, bottom) of analysed plant samples from a plant system exposed to the same conditions of Cd/biochar in FIG. 5. FIG. 7, also abbreviated as FIG. 7, illustrates the cultivation system of Sedum hispanicum in green roof substrates treated or not with 2 types of biochar (TB and AB). FIG. 8, also abbreviated as FIG. 8, illustrates the soil cover of S. hispanicum during 31 days of growth in green roof substrates treated with various percentages (m %) of biochar from six different input materials (COF, PDF, AB W/OG CPS, OS, MDF and TB). FIG. 9, also abbreviated as FIG. 9, illustrates from top to bottom: Average number of nuclei per microliter of carrier fluid; Endoreplication index for growth (EI_{GROWTH}) and for defence (EI_{DEFENCE}) of the analysed plant samples from plant systems exposed to the various percentages (m %) of biochar shown in FIG. 8. FIG. 10, also abbreviated as FIG. 10, illustrates the determination of the ploidy level tipping point T for A. thaliana exposed to a Cd concentration in the range 0-50 μM Cd. FIG. 11, also abbreviated as FIG. 11, illustrates the determination of the ploidy level tipping point T for A. thaliana exposed to a Cd concentration in the range 1-10 μM Cd, wherein the sample is a first leaf of the plant system. FIG. 12, also abbreviated as FIG. 12, illustrates the determination of the ploidy level tipping point T for A. thaliana exposed to a Cd concentration in the range 1-10 μM Cd, wherein the sample is a third leaf of the plant system. FIG. 13, also abbreviated as FIG. 13, illustrates the determination of the ploidy level tipping point T for Dianthus caryophyllus seedlings.

FIG. 14, also abbreviated as FIG. 14, which is divided in FIGS. 14A and B, illustrates from left to right: Average number of nuclei per microliter of carrier fluid; Endoreplication index (EI), for defence (EI_{DEFENCE}) and for growth (EI_{GROWTH}) of the analysed plant samples from different plant systems exposed to the various treatments.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous. When describing the compounds of the invention, the terms used are to be construed in accordance with the following definitions, unless a context dictates otherwise.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

In a first aspect, the present invention relates to a method for determining a toxicity and/or growth promotion effect of any one of a treatment or compound, comprising the steps of:
a) providing at least one plant system or parts thereof comprising at least one cell which ploidy is provided to change on exposure to the any one of a treatment or compound;
b) exposing the plant system or parts thereof to the treatment or compound;
c) providing at least a first and/or a second series of one or more ploidy levels, including e.g. all ploidy levels, of the nuclei in a sample of the exposed plant system or parts thereof, wherein
   the first series is calculated from a basic ploidy level up to, and including, a ploidy level tipping point T corresponding to an arrest/inhibition towards higher ploidy levels for the sample; and
   the second series is calculated from, and excluding, the ploidy level tipping point T to the highest ploidy level observed for the plant system; and
d) determining the toxicity and/or growth promotion effect of the treatment or compound in the plant system based on the first series and/or second series of ploidy levels provided at step c).

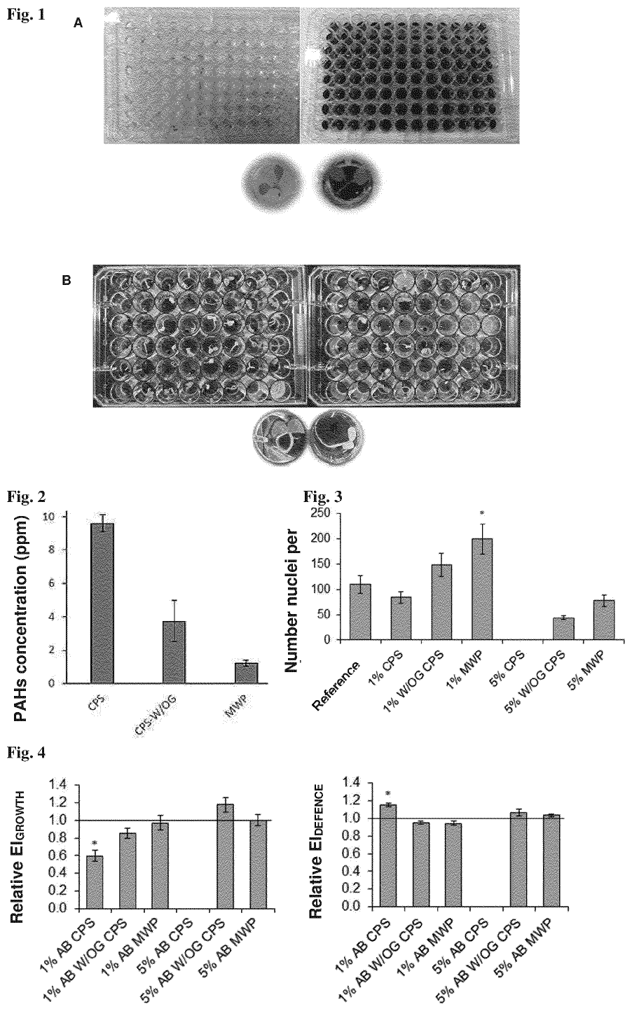

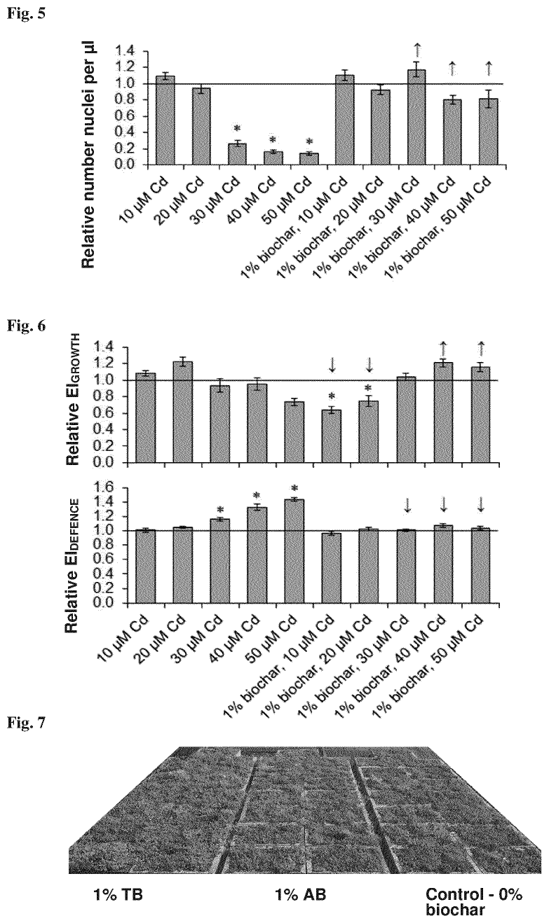

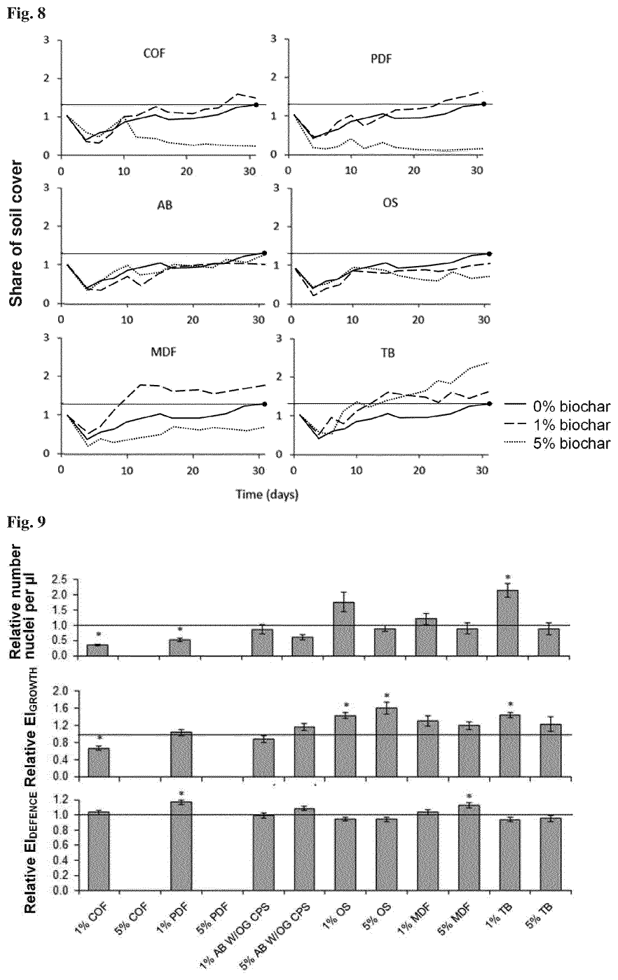

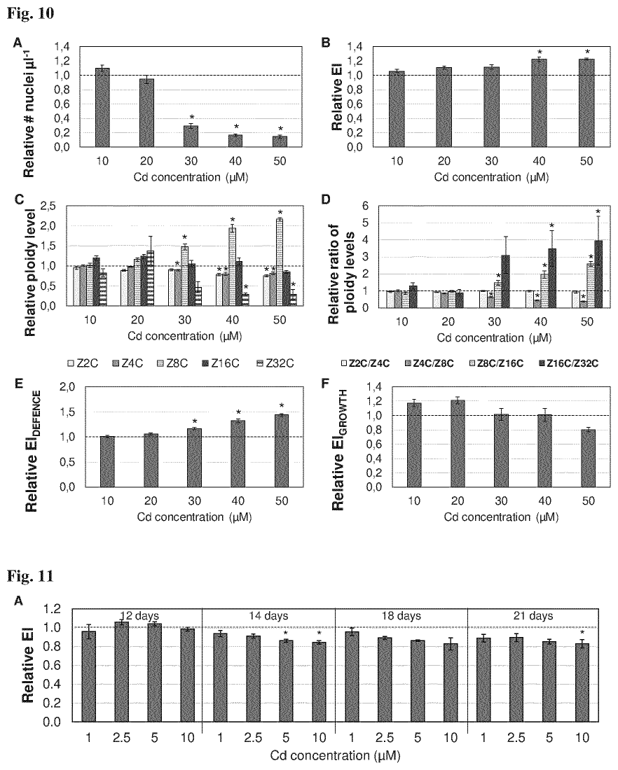

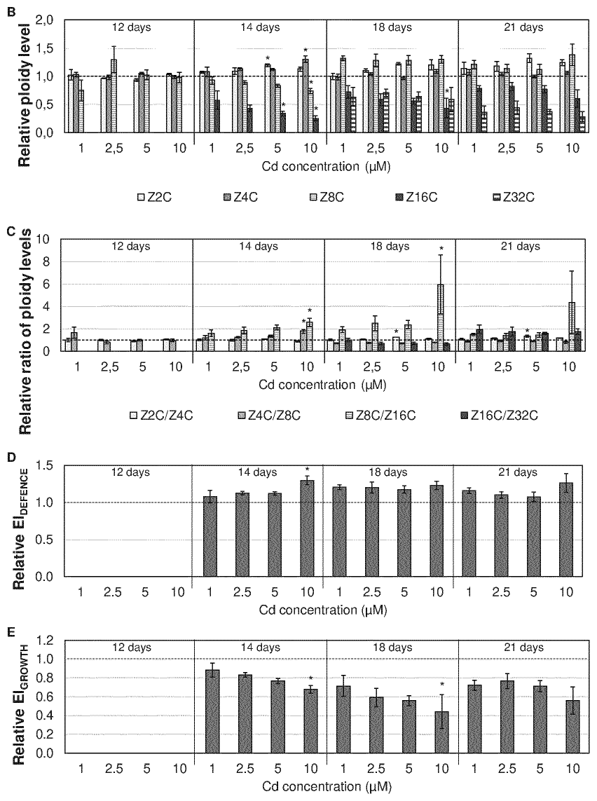

An advantage of the first aspect of the present invention is that it allows to determine of the effects of a treatment or compound on plant responses in a fast way and independently of treatment or compound's physico-chemical parameters or plant system phenotypical analyses. Further, an advantage of the present embodiment is that the effect a treatment or compound has on a plant system can be determined accurately in a reduced amount of time. The method according to the present invention is particularly useful at determining the effect a compound or treatment has on a plant system, which is subjected to said treatment or compound. More specifically, the method according to the present invention allows to determine toxicity and/or growth promotion effect of said compound or treatment on said plant system. In particular, by means of the method according to the present invention it is possible to distinguish on the one hand if the effect provided by said treatment or compound has a growth promoting effect wherein plant growth is supported or an homeostatic effect occurs, wherein the plant system resists or acclimates to change, or on the other hand if the treatment or compound has a defence or stress effect, wherein the plant is in a situation and somehow suffers from the presence of said treatment or compound. The plant system or parts thereof provided at step a) comprises at least one plant cell which ploidy of said cell is provided to change on exposure of the plant or parts thereof to the said treatment or compound. In other words, the present invention allows to distinguish between situations where plants can successfully perform endoreplication to grow and maintain their homeostasis, and where endoreplication is stimulated in defence against stress. In accordance with the present invention, by means of the term “endoreplication”, or “endocycling”, or also known as “endoreduplication”, reference is made to an alternative cell cycle to normal cell division in which the DNA doubles but the cell does not divide. In normal cell division (normal cell cycle), the DNA is first doubled in the S phase and then the cell divides into 2 identical cells in the M phase. The amount of DNA is therefore the same in each daughter cell. In endoreplication, the DNA is doubled in the S phase but this step is not followed by an M phase with cell division. As a result, the DNA remains in the cell to double extent. This process can be repeated several times and such endocycles thus result in endopolyploidy (i.e., DNA replication in cells). Therefore, endoreplication includes duplication of the genome without cell division (i.e., an endocycle), resulting in polyploid cells with multiple DNA copies. Although endoreplication is involved in normal plant growth and development, it is often influenced by stress conditions such as soil quality. In both plants and animals, a shift from normal cell division to endoreplication has been shown to be a conserved method to maintain homeostasis despite stressful conditions. The ploidy level of the nuclei is therefore also a measure of plant stress (i.e., the extent to which the plant can or must defend itself in order to acclimate to the surrounding environment). In particular, in accordance with the first aspect of the method of the present invention, at step a) a plant system or parts thereof, such as leaves, or cells, is provided. In accordance with the present invention, by means of the term “plant system”, reference is made to a plant, and parts thereof, such as cells from said plant. The method according to the present invention can be applied to any plant. In accordance with a further embodiment of the present invention, at step a) the plant system is selected from: Arabidopsis thaliana, Dianthus caryophyllus, . . . preferably Arabidopsis thaliana. It has been found that plant systems in accordance with the present embodiment provide for satisfactory results and are easy to setup. In accordance with a further embodiment of the present invention, at step a) the plant system is provided in a microtiter plate. The microtiter plate can be adapted to grow plant systems of different dimensions. An advantage of the present embodiment is that only a limited space is required to test or screen multiple conditions. Based on the dimensions of the plant system provided, the microtiter plate can be adapted to allow cultivation of e.g. plant seedlings of different size e.g. plants with large seeds are cultivated in a 48, 24 or 12-well microtiter plate instead of a 96-well microtiter plate, also known as 96-well system, which is particularly advantageous with A. thaliana. For example, FIG. 1A illustrates photos of A. thaliana of 7 days old, grown in liquid growth medium in a 96-well microtiter plate without (left) and with (right) addition of biochar whereas FIG. 1B illustrates photos of D. caryophyllus of 9 days old, grown in liquid growth medium in a 48-well microtiter plate without (left) and with (right) addition of 30 UM cadmium (Cd). The plant system at step a), in accordance with a further embodiment of the present invention, can be provided in a growth matrix. In accordance with the present invention, by means of the term “growth matrix”, reference is made to a material or liquid in which a plant or parts thereof is grown, such as soil, substrate (e.g. peat, sand, perlite, rockwool, paper), liquid-based growth medium (e.g. hydroponics, agar, microtiter plate), . . . . Various nutrient solutions are suitable to be used in accordance with the present invention, e.g. water, (modified) Hoagland, Murashige and Skoog medium (MS), Gamborg B5 medium, . . . . The present invention can be applied independently of the growth matrix (type of soil, substrate, liquid-based growth medium) to e.g. make a correlation between the quality of a compound or substance e.g. biochar, as an additive on the basis of its toxicity and/or growth promotion on plants (Poorter et al., 2012). The method according to the present invention further comprises the step b) of exposing the plant system or parts thereof to the treatment or compound. In accordance with the present invention, by means of the term “treatment”, reference is made to the use of one or more of a compound or method e.g. irradiation, lack of nutrients, biochar addition, . . . to improve (e.g. to cure a disease) or worsen the condition of an organism e.g. a plant. In accordance with the present invention, by means of the term “compound”, reference is made to a chemical composition e.g. toxic substances (metals, organic pollutants, pesticides, . . . ), nutrients (salts, N, P, . . . ), growth-promoting substances (hormones, micro-organisms, . . . ), biotic stress (pathogens, . . . ). In other words, reference is made to any biological, physical or chemical composition or mixtures that can be administered to a plant system. As previously explained, either the treatment or the compound can have an effect on the plant. For example, the treatment or compound can have e.g. a growth promotion effect, homeostatic effect, toxicity effect, . . . . The effect said treatment or compound can have on the plant system depends on both the plant model used and the type of treatment/compound. In accordance with a further embodiment of the present invention, at step b) the compound is a carbonized material, such as conventional pyrolysis (CPS) carbonized material, conventional pyrolysis including a washing step and degassing after grinding (CPS-W/OG) obtained carbonized material or microwave pyrolysis (MWP) obtained carbonized material. In accordance with the present invention, by means of the term “carbonized material”, also referred to as biochar or active carbon, reference is made to a solid product rich in carbon, obtainable for example from the carbonization of organic mass, especially organic biomass (biochar). Biochar is usually created by pyrolysis of organic biomass with a temperature between 400 and 700° C. in the absence of oxygen. Upon a steam activation step, active carbon is produced from biochar. In accordance with the present invention, by means of the term “toxicity”, reference is made to the degree to which a substance or mixtures thereof, treatment or organism damages an organism e.g. a plant. In other words, with the term “toxicity”, reference is made to the measurement of the dosage needed of a particular substance or mixtures thereof, treatment or organism to damage an organism e.g. a plant. In accordance with the present invention, by means of the term “growth promotion”, reference is made to the ability of a compound or treatment or organism to support the growth of an organism e.g. a plant. The plant system at step a), in accordance with a further embodiment of the present invention, can be provided in a growth matrix to which the treatment or compound of step b) is applied. For example, the compound can be provided in the soil where the plant system is grown. The method according to the present invention further comprises the step c) of providing at least a first and/or a second series of one or more ploidy levels of the nuclei in a sample of the exposed plant system or parts thereof, wherein the first series is calculated from a basic ploidy level up to, and including, a ploidy level tipping point T corresponding to an arrest/inhibition towards higher ploidy levels for the sample, and the second series is calculated from, and excluding, the ploidy level tipping point T to the highest ploidy level observed for the plant system. In accordance with the present invention, by means of the term “ploidy level”, reference is made to the number of copies of DNA present in the nucleus. The ploidy level of an individual cell is defined as P^{y}C with y being a non-negative integer referring to the number of cell cycles, for which y≤1, or the number of endocycles plus 1, for which y>1. For example, for P^{y}C equals to 2^{y}C, for y=0 corresponds 2° C. equals 1C (meiosis), for y=1 corresponds 21C equals 2C (mitosis), for y=2 corresponds 22C equals to 4C (1+1 endocycle), for y equals 3 corresponds 23C equals to 8C (1+2 endocycles) . . . . By means of the term “basic ploidy level”, indicated by P, reference is made to the number of sets of homologous chromosomes (n) present in that organism. Organisms can be described according to their basic ploidy level: haploid (1 set, P=1, denoted as n), diploid (2 sets, 2n), triploid (3 sets, 3n), tetraploid (4 sets, 4n), . . . . A genome is a complete single set of chromosomes. In a non-dividing cell with a basic ploidy level, each chromosome represents a copy of a DNA helix (C) and, for example, 2n equals 2C. The latter is referred to as 2C where C stands for the haploid DNA content (i.e., the number of copies of the genome). Haploid organisms such as certain mosses contain one copy of each chromosome (i.e., genome) while diploid organisms such as A. thaliana contain 2 copies and tetraploid organisms such as D. caryophyllus contain 4 copies of each chromosome. The highest ploidy level that can be reached by plant cells via endoreplication is species-dependent. For example, the highest number of endocycles detected in diploid (2C) A. thaliana cells is 4, resulting in a maximum ploidy level of 32C (P^{y}C=2⁴⁺¹C=2⁵C=32C). The inventors have found out that in order to discern the effect of a compound or treatment to a plant system, and more specifically if said effect is a homeostatic/growth effect or a defence/stress effect, it is required to consider all or only some, e.g. two or more, of the ploidy levels which can be achieved by a plant system. The ploidy levels that have to be taken into account to determine if an effect can be considered as a defence/stress effect are the ploidy levels from a basic ploidy level up to, and including, a ploidy level tipping point T, which series of ploidy levels, referred to as first series of ploidy levels, allows to discern if said effect is a defence/stress effect. For example, in case the plant system is A. thaliana, the maximum ploidy level is 32C, whilst the basic ploidy level is 2C, and for a specific plant system (e.g. type of plant and age) or parts used thereof, the tipping point ploidy level T is for example 8C. The first series of ploidy levels comprises the ploidy levels 2C, 4C and 8C. Therefore, only ploidy levels 2C, 4C and 8C are to be utilized to discern the defence/stress effect of said compound. The inventors have found out that in order to discern the effect of a compound or treatment to a plant system, and more specifically if said effect is a homeostatic/growth effect or a defence/stress effect, it is required to consider all or only some of the ploidy levels which can be achieved by a plant system. The ploidy levels that have to be taken into account to determine if an effect can be considered as a homeostatic/growth effect are the ploidy levels from, and excluding, the ploidy level tipping point T to the highest ploidy level observed for the plant system, which series of ploidy levels, referred to as second series of ploidy levels, allows to discern if said effect is a homeostatic or growth inducing effect. For example, in case the plant system is A. thaliana, the maximum ploidy level is 32C, whilst the basic ploidy level is 2C. The second series of ploidy levels comprises the ploidy levels 16C and 32C. Therefore, only ploidy levels 16C and 32C are to be utilized to discern the homeostatic/growth inducing effect of said compound. Therefore, in accordance with the present invention, either the first series and/or the second series of ploidy levels provided at step c) is further used at step d), wherein the toxicity and/or growth promotion effect of the treatment or compound in the plant system is determined based on said first series and/or second series of ploidy levels. For example, the first series and/or the second series can be used to calculate the endoreplication indices EI_{DEFENCE} and EI_{GROWTH} in accordance with an embodiment of the present invention, by means of only the ploidy level abundances. When the abundances of the ploidy level tipping point T or higher ploidy levels increase relative to the control (without treatment or compound), this can also give an indication or the toxicity or growth promotion effect, respectively. By means of only the relative abundance ratio with respect to its subsequent ploidy level, when the ratio of the ploidy level tipping point T increases or does not increase relative to the control, this can also give an indication or the toxicity or growth promotion effect, respectively. Further, the relative concentration of the nuclei can also be used to determine the toxicity and/or growth promotion effect. The ploidy level tipping point T, which corresponds to the ploidy level for which an arrest/inhibition towards higher ploidy levels has been observed, is dependent on both the plant system utilized and plant parts analysed. In accordance with the present invention, the ploidy level tipping point T, corresponds to the ploidy level P^{a}C, wherein a is equal to log_{P}(T), in other words, it is the logarithm base P of the tipping point ploidy level. For example, based on an example of the present invention in the plant system A. thaliana, the arrest/inhibition towards higher ploidy levels can be determined by identifying the dose-dependent increase of a specific ploidy level relative to a control (T) in which case the higher ploidy levels do not increase relative to a control, without the need of determining the relative abundance ratio. For example, if it is observed that the ploidy level tipping point T, is equal to P^{a}C, for a specific plant system having a basic ploidy level of 2C and exposed to a compound is 8C, cells of said plant system underwent 2 endocycles prior to and including an arrest/inhibition towards higher ploidy levels, 8C=2³C and therefore a=3, wherein a=log_{P}(T)=log₂(8C)=3, wherein C is the abbreviation for DNA copies, T is the ploidy level tipping point and P is the basic ploidy level of the plant system. In accordance with an embodiment of the present invention, the ploidy level tipping point T, or simply tipping point T, is defined as the lowest ploidy level for which its relative abundance ratio with respect to its subsequent ploidy level, shows a dose-dependent increase with the treatment or compound within a certain range. It has been found to determine of the tipping point T, the present embodiment provides for the most reliable and reproducible results. For example, FIG. 10 illustrates various measurements pertaining to the relative ploidy levels of a plant system of A. thaliana. The plant system has been exposed to cadmium (Cd) at various concentrations (dose). The plant system underwent both regular cell division and endoreplication. FIG. 10C shows that for increasing concentrations of Cd (10 μM to 50 μM), the relative amount of the nuclei containing 8C of a sample of said plant system increased. More specifically, it can be seen that the fraction of nuclei for the ploidy level 8C increase gradually with the concentration of Cd, from 10 UM to 50 μM, which is not the case for the nuclei with higher ploidy levels (i.e., 16C and 32C). In order to determine from which concentration of Cd, Cd would start to have a negative effect onto the plant system (a defence/stress effect), the inventors have surprisingly found that the determination of the tipping point T is beneficial. In accordance with the present embodiment, the ploidy level tipping point T is the lowest ploidy level for which its relative abundance ratio with respect to its subsequent ploidy level, shows a dose-dependent increase with the treatment or compound within a certain range. In this case, FIG. 10D, shows the relative abundances 2C/4C, 4C/8C, 8C/16C and 16C/32C. It has been found that the ploidy level tipping point T is in the present case the lowest ploidy level (8C), for which the relative abundance ratio (8C/16C) shows a dose-dependent increase with respect to the control (dashed line parallel to the x axis) for the range 0-50 μM Cd. This range is determined between 0 and a toxic but sublethal dose within the experimental timeframe (e.g. 50 μM). Therefore, by means of the present embodiment, the ploidy level corresponding to an arrest/inhibition towards higher ploidy levels (T) can be determined. For a second example, FIG. 13 illustrates various measurements pertaining to the relative ploidy levels of a plant system of D. caryophyllus. The plant system has been exposed to Cd at various concentrations (dose). The plant system underwent both regular cell division and endoreplication. FIG. 13A shows that for increasing concentrations of Cd (10 μM to 50 μM), the relative amount of the nuclei containing 4C of a sample of said plant system increased. More specifically, it can be seen that the fraction of nuclei for the ploidy level 4C increases gradually with the concentration of Cd, from 10 UM to 50 μM, which is not the case for the nuclei with higher ploidy levels (i.e., 8C, 16C and 32C). In order to determine from which concentration of Cd, Cd would start to have a negative effect onto the plant system (a defence/stress effect), the inventors have surprisingly found that the determination of the tipping point T is beneficial.

In accordance with the present embodiment, the ploidy level tipping point T is the lowest ploidy level for which its relative abundance ratio with respect to its subsequent ploidy level, shows a dose-dependent increase with the treatment or compound within a certain range. In this case, FIG. 13B shows the relative abundances 4C/8C, 8C/16C and 16C/32C. It has been found that the ploidy level tipping point T is in the present case the lowest ploidy level (4C), for which the relative abundance ratio (4C/8C) shows a dose-dependent increase with respect to the control (dashed line parallel to the x axis) for the range 0-50 μM Cd. Therefore, by means of the present embodiment, the ploidy level corresponding to an arrest/inhibition towards higher ploidy levels (T) can be determined.

In accordance with a further embodiment of the present invention, at step d) the toxicity and/or growth promotion effect of the treatment or compound is determined based on data measured by means of flow cytometry. In accordance with the present invention, by means of the term “flow cytometry”, reference is made to a technique used to detect and measure physical and chemical characteristics of a population of cells or particles. A flow cytometer is used to count and study microscopic particles in a flowing liquid (i.e., carrier fluid). In accordance with the present invention, a flow cytometer is used to register the amount of DNA per nucleus. The higher the signal, the more DNA is present in the measured nucleus, and the higher the ploidy level of the plant cell. Flow cytometry uses the light properties scattered from cells or particles for identification or quantitative measurement of physical properties.

An advantage of the present embodiment is that the technical implementation is simple and does not influence the results' interpretation, making the results very robust and reliable. Further, in accordance with the present embodiment the screening is very sensitive and can be performed at very low concentrations of treatments or compounds and low biomass is needed from the plant or plants thereof, which is beneficial to certain applications.

Further, the first aspect of the present invention further comprises step d) of determining the toxicity and/or growth promotion effect of the treatment or compound in the plant system based on the first series and/or second series of ploidy levels provided at step c).

In accordance with an embodiment of the present invention, step d) of determining the toxicity and/or growth promotion effect is based on one or more of an endoreplication index (EI) calculated in accordance with formula (I) and/or formula (II)

$${EI}_{DEFENCE} = {\sum }_{y = 0}^{y = a} ( {x}_{y} \times {Z}_{y} ) \qquad ( I )$$

$${EI}_{GROWTH} = {\sum }_{y = a + 1}^{y = k} ( {x}_{y} \times {Z}_{y} ) \qquad ( II )$$

Formula (I) and formula (II) are particularly useful at determining the toxicity and/or growth promotion effect of the treatment or compound in the plant system based on said treatment or compound it has been exposed to. Endoreplication indices EI_{DEFENCE} and EI_{GROWTH} denote the degree of endopolyploidization for the ploidy levels relevant to determining a defence/stress effect on the one hand, and a growth promotion/homeostatic effect on the other hand. The endoreplication index calculated in accordance with the state of the art (i.e., EI) includes all ploidy levels available to the plant system (Hendrix et al., 2018) and hence is not relevant for distinguishing said growth promotion/homeostatic effect on the one hand, and defence/stress effect on the other hand. In accordance with the present invention, the indices EI_{DEFENCE} and EI_{GROWTH} are calculated taking into account part and not all the ploidy levels available to the plant system, more specifically, EI_{DEFENCE} and EI_{GROWTH} are calculated taking into account said first series and second series of ploidy levels. More specifically, EI_{DEFENCE}, also referred to as defence endoreplication index, denotes the degree of endopolyploidization for the ploidy levels belonging to the first series provided at step c), whereas EI_{GROWTH} also referred to as growth endoreplication index, denotes the degree of endopolyploidization for the ploidy levels belonging to the second series provided at step c). In accordance with a further embodiment of the present invention, at step d) the toxicity effect is determined based on the first series provided at step c) or EI_{DEFENCE}, and/or the growth promotion effect is determined based on the second series provided at step c) or EI_{GROWTH}. In accordance with a further embodiment, the toxicity effect on the plant system is determined by means of EI_{DEFENCE} of the plant system being higher than a EI_{DEFENCE} for a control, and/or the growth promotion effect on the plant system is determined by means of EI_{GROWTH} for the plant system being higher than a EI_{GROWTH} for a control. In other words, the presence of an effect of the treatment/compound is established by comparing any one of the two endoreplication indices with the ones obtained for a control, which control can be for example a plant system whereby no treatment/compound is provided, or a mock treatment/compound. Formula (I) and (II) are defined by means of the following indices/variables. By means of the index y, reference is made to the index of each summation and a non-negative integer referring to the number of cell cycles, meaning mitosis and meiosis, for which y $1, or the number of endocycles plus 1, for which y>1. For EI_{DEFENCE}, the start of the summation corresponds with the number 0 and ends at a, wherein a is equal to log_{P}(T), wherein T is the ploidy level tipping point T, equals to P^{a}C, corresponding to an arrest/inhibition towards higher ploidy levels. For EI_{GROWTH}, the index of the summation starts at a+1 and ends at k, which is the highest ploidy level observed for the plant system. Further, in formula (I) and (II), by means of the term x_{y}, reference is made to an element of the real numbers. In other words, x_{y} can be a weight factor correlated to y, in which x_{y=0}≤x_{y=1}≤ . . . ≤x_{y=a} for EI_{DEFENCE} and x_{y=a+1}≤x_{y=a+2}≤ . . . ≤x_{y=k} for EI_{GROWTH}. For example, x_{y} can be a number proportional to the index of each summation y, or can be selected based on the number of endocycles needed to reach the corresponding ploidy level or on another biological or mathematical model. Further, by means of the term Z_{y}, reference is made to the fraction of nuclei in a sample of the plant system or parts thereof of the represented ploidy level P^{y} DNA copies (C), wherein P is the basic ploidy level of said plant system. Term Z_{y} could be multiplied to a normalization factor, which can be included in x_{y}. For example, here below are shown the calculations of formula (I) and formula (II) for the plant system of Example 4, wherein A. thaliana seedlings are cultivated in MS medium, which system has a tipping point T equal to 8C, and therefore a=3.

$${EI}_{DEFENCE} = {\sum }_{y = 0}^{y = a} ( {x}_{y} \times {Z}_{y} ) = {\sum }_{y = 0}^{y = 3} ( {x}_{y} \times {Z}_{y} ) = 0 \times {Z}_{0} + 0 \times {Z}_{1} + 1 \times {Z}_{2} + 2 \times {Z}_{3} \\ {EI}_{GROWTH} = {\sum }_{y = a + 1}^{y = k} ( {x}_{y} \times {Z}_{y} ) = {\sum }_{y = 4}^{y = 5} ( {x}_{y} \times {Z}_{y} ) = 3 \times {Z}_{4} + 4 \times {Z}_{5}$$

For example, in order to use the formula above, e.g. the fraction of the nuclei can be measured for each ploidy level e.g. 2C, 4C, 8C, 16C, 32C by means of flow cytometry and then incorporated in the formulas (I) and (II) above. For example, in case the fractions of the nuclei for EI_{GROWTH} above are 2.5 for the ploidy level 16C and 1.5 for the ploidy level 32C, EI_{GROWTH} would be equal to 3×Z₄+4×Z₅=3×2.5+4×1.5=7.5+6.0=13.5 Therefore, it should be clear to the skilled in the art that the terms 20, 4C, 8C, 16C, 32C . . . denote the ploidy levels and are therefore indices for the fractions of nuclei Z_{y}. In accordance with a preferred embodiment of the present invention, both EI_{DEFENCE} and EI_{GROWTH} can be calculated. This becomes convenient when for example by means of flow cytometry the various parameters of the sample of the plant system are determined, such as concentration of the nuclei and ploidy levels. In a second aspect, the present invention relates a computer program comprising instructions which, when the program is executed by a computer, causes the computer to carry out the steps c) and/or d) of the method of method according to the present invention. In particular, the computer program can comprise instructions so to calculate formula (I) and/or (II), and/or any of a concentration of the nuclei or ploidy levels in accordance with a first series and second series of ploidy levels. The computer program allows the user to readily derive information pertaining to the growth promotion/homeostatic effect on the one hand (e.g. by means of formula (II)), and defence/stress effect on the other hand (e.g. by means of formula (I)), on the plant system of the compound or treatment screened. Additionally, the computer program can comprise instructions so to calculate the tipping point T, wherein the ploidy level tipping point T is determined as the lowest ploidy level for which its relative abundance ratio with respect to its subsequent ploidy level, shows a dose-dependent increase with the treatment or compound within a certain range.

### EXAMPLES

### Statistics

Statistical analysis was performed using RStudio (version 3.3.1; R Foundation for Statistical Computing, 2016). Normal distribution of the datasets was tested using the Shapiro-Wilk test, and homoscedasticity was evaluated with the Bartlett's test. If necessary, transformations of the datasets were applied. Significant differences were determined using ANOVA test and Tukey correction. If the assumption of normality was not fulfilled, then a non-parametrical ANOVA test (Kruskal-Wallis) and correction with pairwise Wilcoxon rank sum test was applied. All data were tested at a significance level of 0.05. The data presented are the means±the standard error (SE).

### Example 1: Effect of Biochar Obtained Via Different Process Parameters on A. thaliana Growth

This first example shows data from 3 biochar types, each produced with different process parameters. The toxicity of the biochars is first determined by means of a physicochemical analysis. Subsequently, this toxicity is determined in accordance with the method of the present invention, with a flow cytometry analysis.

### Materials and Methods

### Biochar

As input material to produce biochar, a wood mix of category A (i.e., untreated wood waste) and category B (i.e., uncontaminated but treated wood waste) is used (AB wood). After the pyrolysis process, each biochar is finely ground using a planetary ball mill (Retsch S1, Germany) and sieved to a fraction less than 63 μM. Three different biochar types are produced: via conventional pyrolysis (CPS), conventional pyrolysis including a washing step and degassing (24 h at 105° C.) after grinding (CPS-W/OG), and microwave pyrolysis (MWP) (see pyrolysis details in Haeldermans et al., 2019). The concentration of 16 toxic polycyclic aromatic hydrocarbons (PAHs) was determined for each biochar (Hilber et al., 2012).

### Cultivation System

Wild-type A. thaliana plants are grown in sterile liquid growth medium such as ¼ Murashige and Skoog (MS) medium (Murashige & Skoog Basal Salt Mixture, most widely used worldwide for tissue or cell culture of plants) and 0.5% sucrose with the addition of a concentration series of biochar [0, 1 and 5% calculated in percent by mass (m %)]. After adding the growth medium to the 96-well microtiter plate, one sterilized A. thaliana seed is placed in each well with tweezers. The plates are closed with a lid and Parafilm®, and transferred to climate chambers (12 h exposure with 170 μmol·m⁻²·s⁻¹ photosynthetic active radiation, 22/18° C. day/night temperature and 65% relative humidity).

### Flow Cytometer Analysis

After seven days the plants are collected in sets of 4 in an Eppendorf® tube and placed in liquid nitrogen and then stored at −80° C. (biological replicates: n=8). The analysis method is described in Hendrix et al., 2018, with the exception of our newly defined endoreplication indices (EI_{GROWTH} and EI_{DEFENCE}). The samples are chopped into extraction buffer (CyStain® PI Absolute P kit, Sysmex Partec) and filtered with a 50 μm nylon filter (CellTrics®, Sysmex Partec). A staining solution containing RNase A and propidium iodide (CyStain® PI Absolute P kit) is then added to each sample and incubated for at least one hour at 4° C. in the dark. A carrier fluid carries the nuclei through the measuring cell passing the laser, where they are each measured separately (488 nm excitation, 580/30 nm emission, CyFlow® Cube 8 flow cytometer, Sysmex Partec). The forward scatter plot is based on the light intensity of the laser that can be measured through the core. By plotting the fluorescence with respect to the forward scatter plot, a distinction is made between the nuclei and their ploidy level, and other particles (background). Analysis is done using FCS Express 5 software (De Novo Software, USA). In this example, the EI_{GROWTH} is calculated as (3×Z₄+4×Z₅)/100, and the EI_{DEFENCE} is calculated as follows: (1×Z₂+2×Z₃)/100, wherein Z_{y} corresponds to the percentage of the corresponding ploidy level with respect to all measured nuclei. For the present calculations, the tipping point ploidy level T is equal to 8C. The weights x_{y} in the formulas above have been selected because of the number of endocycles.

### Results and Discussion

The 3 different biochar types of AB wood (CPS, CPS-W/OG and MWP) are added to the growth medium of the A. thaliana plants at a percentage (m %) of 0 (control), 1 or 5% biochar to investigate the toxicity or growth promotion of the biochar types. The concentration of PAHs is measured for each type, see FIG. 2, which is significantly higher after conventional pyrolysis (CPS) compared to the other 2 types. The post-treatment (CPS-W/OG) has a clear decreasing effect on the PAH concentration in the biochar, while microwave pyrolysis (MWP) indicates the lowest PAH concentration even without post-treatment. Since PAHs are toxic to living organisms, this physicochemical analysis suggests that the MWP biochar will give the best result on plant growth and stress, followed by the CPS-W/OG and CPS biochar. The A. thaliana plants are exposed to the different conditions in the 96-well system. After seven days, the concentration of nuclei and the ploidy levels are determined by flow cytometry. The decreasing trend in PAHs concentration of the 3 biochar types corresponds to an increasing trend in the number of nuclei per microliter at both biochar percentages, see FIG. 3. FIG. 3 illustrates the average number of nuclei per microliter of carrier fluid of the analysed plant samples. Significance level (one-way ANOVA) relative to the control condition: * p<0.05. This indicates an increasing plant growth (more cell divisions) the less PAHs are present in the biochar and vice versa, the more toxic the biochar, the less well the plants grow. This negative correlation between PAHs and plant growth is translated into a correlation coefficient (r) of −0.61 (p<0.05). An important side note is that higher biochar percentages are often less good for plant growth, a fact that is confirmed in the 96-well system where plant growth is higher with 1% biochar than with 5% biochar. When exposed to 5% of the most toxic CPS biochar with the highest PAH concentration, the plants could not even germinate, hence the missing data. When factoring in ploidy levels, see FIG. 4, the CPS biochar causes significantly decreased EI_{GROWTH} and significantly increased EI_{DEFENCE} in the plants relative to the control condition, both indications of stress/defence. FIG. 4 illustrates endoreplication index for growth/homeostasis (EI_{GROWTH}, left) and for stress/defence (EI_{DEFENCE}, right) of the analysed plant samples, expressed relative to the control condition shown by the horizontal line (1.0). Significance level (one-way ANOVA) relative to the control condition: * p<0.05. Although the other treatments do not result in significant differences, the 1% biochar conditions show an increasing trend in EI_{GROWTH} (r=−0.67, p<0.05) and a decreasing trend in EI_{DEFENCE} (r=0.86, p<0.05) with decreasing PAHs concentration.

### Conclusion

The results of the flow cytometry provide a clear correlation with the predicted toxicity related to the PAH content in the biochar. In summary, PAHs concentration is positively correlated with EI_{DEFENCE} and negatively correlated with the number of nuclei per microliter and EI_{GROWTH}. These results show that the method according to the present invention is a valid alternative to measuring biochar toxicity based on the physicochemical PAH analysis.

### Example 2: Effect of Biochar on the Growth of A. thaliana in the Presence of Cd

This second example shows data from A. thaliana plants exposed to 5 Cd (cadmium) concentrations with or without the addition of AB biochar. Cd toxicity is predicted by our invention by flow cytometry analysis.

### Materials and Methods

### Biochar

Conventional pyrolysis of AB wood is followed by a 3 step post-treatment as described in Example 1, meaning grinding, degassing and sieving. The AB biochar from this example is therefore identical to the CPS-W/OG biochar from Example 1. In the control condition, no biochar is added to the cultivation system used.

A. thaliana

The culture system and flow cytometer analysis are identical to Example 1. Arabidopsis thaliana (A. thaliana) seeds are exposed from sowing to concentrations of 0, 10, 20, 30, 40 and 50 μM Cd (as CdSO₄) with or without addition of 1% biochar. Exposure took ten days instead of seven days to generate sufficient biomass as Cd has an adverse effect on plant growth.

### Results and Discussion

Cadmium is known for its growth-inhibiting effect on plants. Literature has shown that biochar can limit this effect by adsorbing Cd. The first statement (plant growth inhibition) is confirmed by the results shown in FIG. 5. FIG. 5 illustrates the average number of nuclei per microliter of carrier fluid of the analysed plant samples relative to the control. Significance level (Kruskal-Wallis) compared to the control condition (*) and significantly increased (1) compared to the same Cd concentration without biochar, p<0.05). Namely, exposure to Cd concentrations from 30 UM or higher causes a significant decrease in the number of nuclei per microliter (i.e., reduced plant growth) of A. thaliana. This negative correlation between Cd and plant growth is translated into a correlation coefficient of −0.83 (p<0.05). The second proposition (reduction of Cd-induced growth inhibition by biochar) is also confirmed as addition of 1% AB biochar completely abolishes the plant growth inhibitory effect upon exposure to 30, 40 and 50 μM Cd, see FIG. 5. When factoring in ploidy levels, see FIG. 6, Cd shows a negative correlation with EI_{GROWTH} (r=−0.39, p<0.05) and a positive correlation with EI_{DEFENCE} (r=0.87, p<0.05). Exposure to 30 μM Cd or more results in significant and dose-dependent increases in EI_{DEFENCE}, indicating stress/defence against the Cd present. FIG. 6 illustrates endoreplication index for growth/homeostasis (EI_{GROWTH}, top) and for stress/defence (EI_{DEFENCE}, bottom) of the analysed plant samples, expressed relative to the control condition shown by the horizontal line (1.0). Significance level (two-way ANOVA) over the control condition (*) and significantly increased (↑) or decreased (↓) over the same Cd concentration without biochar, p<0.05). Addition of biochar prevents stress to the plants, which is confirmed by the recovery in growth, see FIG. 5, and EI_{DEFENCE}, see FIG. 6, to the same level as the control condition. Remarkably, addition of biochar reverses the negative correlation between Cd and EI_{GROWTH} to a positive correlation (r=0.81, p<0.05), see FIG. 6. This indicates that biochar positively influences the plants' ability to continue their endoreplication to the 16C and 32C ploidy level, potentially limiting Cd-induced DNA damage and maintaining homeostasis.

### Conclusion

The results of the flow cytometry in the plants provide a clear correlation with the predicted Cd toxicity. In summary, the Cd concentration is positively correlated to EI_{DEFENCE} and negatively correlated to the number of nuclei per microliter and EI_{GROWTH}. Addition of biochar prevents the adverse effects of Cd and ensures that the plants react as under control conditions. Only with regard to the EI_{GROWTH}, the addition of biochar provides a positive correlation with Cd. This indicates that biochar supports the plants with a successful defence against Cd, which is not needed under low concentrations (10-20 μM Cd) but is needed at higher concentrations (30-50 μM Cd). An increased ploidy level ensures that the integrity of the genome can be maintained, even in the case of DNA damage. These results support the use of the method according to the present invention as a valid alternative to predict the toxicity of Cd and the beneficial effect of biochar.

### Example 3: Input Materials Influence the Biochar Quality

The present example shows data from six biochar types that were produced with the same process parameters but different input materials. The toxicity of the biochar types is first determined by means of a physicochemical analysis, followed by a green roof experiment. Finally, this toxicity is predicted by our new analysis method using flow cytometry.

### Materials and Method

### Biochar

Biochar types from six different input materials are produced via conventional pyrolysis (CPS) without additional post-treatment as described in Example 1: coffee grounds (COF), Palm Date Fronds (PDF), wood category AB (AB), olive kernels (OS), Medium-Density Fiberboard (MDF) and Tree Bark (TB) (for your information: the AB biochar is identical to the CPS biochar from Example 1). Standard physicochemical measurements are performed on each biochar: PAHs, pH, conductivity and water holding capacity (WHC). In the control condition, no biochar is added to the cultivation system used.

Sedum hispanicum—Cultivation System and Ground Cover

Sedum hispanicum cuttings are planted in a green roof substrate (clay, lava, green compost, 5% organic matter) with or without the addition of 1 and 5% biochar. The plants are grown in a controlled greenhouse (20-24° C., 65% relative humidity) for 31 days and watered every 2 or 3 days. For Sedum hispanicum, plant growth was evaluated by land cover using the automated method based on Guijarro et al., 2011. Specifically, green pixels from photographs are quantified. It is corrected for the background and the initial ground cover on day 1.

A. thaliana

The culture system and flow cytometer analysis are identical to Example 1.

### Results and Discussion

Six biochar types from six different organic residual flows are added to the green roof substrate for S. hispanicum and to the growth medium of A. thaliana at a percentage (m %) of 0 (control), 1 or 5% to compare the toxicity or growth promotion of the biochar types in both culture systems. On the basis of the physicochemical analyses, see Table 1 below, the biochar types are divided into 2 promising and 4 less suitable types. Table 1 shows PAHs, pH, conductivity and WHC for the six biochar types. The target value for these parameters is also shown at the bottom. The AB biochar is less suitable because of its high PAH content. The COF and PDF biochar types have a relatively high PAH content, but their high conductivity is especially disadvantageous because this means a strong leaching of nutrients. The OS biochar has a very low water holding capacity (WHC), which makes it uninteresting as a plant growth additive in soil/substrate. Both the MDF and the TB biochars score well on each parameter, so they belong to the promising biochar types. See Table 1 here below:

| Biochar | PAH content | | conductivity | |
|---|---|---|---|---|
| type | (ppm) | pH | (μS/cm) | WHC (%) |
| COF | 5.89 | 9.3 | 800 | 398 |
| PDF | 5.35 | 7.9 | 1175 | 376 |
| AB | 9.61 | 7.5 | 583 | 306 |
| OS | 0.40 | 8.3 | 650 | 70 |
| MDF | 0.91 | 7.4 | 575 | 332 |
| TB | 1.54 | 7.6 | 581 | 231 |
| Target | As low as | 7.35 | 580 | At least |
| values | possible | | | 100% |

Experiment with Green Roof Substrate in the Greenhouse

During the greenhouse experiment, S. hispanicum is cultivated in green roof substrates, see experimental design in FIG. 7, and photographed per treatment for 31 days. FIG. 8 shows the share of soil cover as a function of the time after planting for the various treatments. More precisely, FIG. 8 illustrates soil cover of S. hispanicum during 31 days of growth in green roof substrates treated with 0% (full line), 1% (dashed line) and 5% biochar (dotted line) from six different input materials. The straight horizontal line represents the final coverage when no biochar was added to the substrate. Both the final coverage achieved by the control condition after 31 days and the speed with which this is reached are indicators for determining the biochar quality. In practice, the faster the soil is covered, the less the system will experience problems such as evaporation, leaching and weeds. The 4 less suitable biochar types, classified according to the physico-chemical analyses, are confirmed in the green roof experiment. The addition of 1% COF and PDF biochars, with especially a strong leaching of nutrients, does not provide a substantial improvement in the coverage level compared to the control condition. The addition of 5% biochar clearly indicates a sharp drop in the soil cover, indicating stress for the plants. This can possibly be salt stress due to the leaching of salts. The AB biochar with a high content of PAHs does not improve the control condition without biochar at both biochar percentages. If this biochar were to have positive effects on plant growth, they would appear to be nullified by the toxic PAHs. The OS biochar with very low WHC also does not give any advantage, the soil cover ratio is even lower with both biochar percentages than with the control condition.

Although both the MDF and the TB biochar are among the promising biochar types, it is only the TB biochar that offers clear benefits at both biochar percentages. Namely, the condition with the lowest TB biochar percentage reaches the final coverage of the control condition after only 12 days instead of 31 days, 5% TB biochar already reaches this after 10 days. With the MDF biochar, on the other hand, only 1% admixture provides an accelerated soil cover (after 9 days instead of 31 days), while 5% admixture clearly does worse than the control condition. Only this last finding of a negative effect with 5% MDF biochar could not be derived from the physicochemical analyses, but for the other conditions the expectations based on the physicochemical analyses are confirmed by the green roof experiment. In addition, this experiment confirms the importance of a screening with a concentration series.

Rapid Screening in the Laboratory in Accordance with the Present Invention

The A. thaliana plants are exposed to the different conditions in the 96-well system. After seven days, the concentration of nuclei and the ploidy level is determined by flow cytometry, see FIG. 9. FIG. 9 illustrates from top to bottom: Average number of nuclei per microliter of carrier fluid; Endoreplication index for growth (EI_{GROWTH}) and for defence (EI_{DEFENCE}) of the analysed plant samples. All values are expressed relative to the control condition shown by the horizontal line (1.0). Significance level (one-way ANOVA) relative to the control condition: * p<0.05. Three of the 4 less suitable biochar types, classified according to the physicochemical analyses and the green roof experiment, are confirmed by the method in accordance with the present invention (also referred to herein as rapid screening). The COF and PDF biochars with a particularly strong nutrient leaching have a negative influence on the germination and growth of seedlings.

Seeds could not germinate after the addition of 5% biochar and the addition of 1% biochar resulted in a significant decrease in the number of nuclei per microliter. The COF biochar shows a significant decrease in EI_{GROWTH}. The PDF biochar, on the other hand, shows a significant increase in EI_{DEFENCE}. All these results indicate stress (possibly salt stress) for the plants. Analogous to the green roof experiment, the AB biochar with high PAH content at both biochar percentages showed no improvement according to the flow cytometer data compared to the control condition without biochar.

Apart from a very low WHC, the OS biochar does not show any negative characteristics according to the physicochemical analysis. Since the 96-well system uses a liquid growth medium, the WHC of biochar does not affect the results of the screening test, so this biochar does very well in the screening as indicated by significant increases in EI_{GROWTH}. From this we conclude that biochar types that are promising according to the screening with A. thaliana must always be analysed for their WHC, which will be decisive depending on the matrices in which the biochar will be used. The MDF biochar shows promise according to the physicochemical analyses, but in a higher percentage it is toxic according to the green roof experiment. According to the 96-well screening, little difference can be seen with the control condition, but the increased toxicity at 5% MDF biochar is translated into a significant increase in EI_{DEFENCE}. In contrast to the physicochemical analyses, the flow cytometer data can therefore identify this toxicity. The most promising TB biochar according to the above data is also classified as the most promising according to the 96-well screening. Addition of 1% TB biochar shows a significant increase in the number of nuclei per microliter and EI_{GROWTH}, while the EI_{DEFENCE} does not change. These effects are still observable with the addition of 5% biochar, although no longer significant, but there is certainly no indication of toxicity.

### Conclusion

The TB biochar scores very well according to the 3 analyses: physicochemical, green cover of S. hispanicum and flow cytometry via the rapid screening with A. thaliana. The MDF biochar also scores well, but to a lesser extent than the TB biochar at higher percentages, which is only demonstrated by the green roof experiment and the rapid screening. This indicates that the rapid screening has a higher sensitivity than the physicochemical analysis. The low expectations of the COF, PDF and AB biochar are confirmed by all analyses of this experiment. The OS biochar teaches us that according to the screening test, a promising biochar must always be analysed for its WHC because this property cannot be included in the liquid growth medium of the screening. Thus, after seven days, the rapid screening can confirm the results of the 31-day long-term experiment. The goal of offering a rapid screening, independent of the growth matrix, for toxicity or growth promotion of components is supported by these results. Based on the current data, we can confirm our hypothesis that the share of the different ploidy levels acts as a tipping point between growth/homeostasis and stress/defence. This difference can be statistically demonstrated in accordance with the method of the present invention, wherein 2 new endoreplication indices, EI_{GROWTH} and EI_{DEFENCE} are used. Further, the screening system (96-well system) in accordance with the present invention is able to replace lengthy and labour-intensive experiments to predict the toxic or growth-promoting effect of biochar. Moreover, this prediction is independent of the growth matrix into which the biochar is mixed.

### Example 4: A. thaliana Seedlings Cultivated in MS Medium Exposed to Cadmium-Determination of Ploidy Level Tipping Point T

Seeds of A. thaliana plants are sown in MS medium and exposed to a concentration range of the toxic metal cadmium (Cd). The plants show a dose-dependent growth retardation, reflected in a decreasing concentration of nuclei, see FIG. 10A, and a dose-dependent induction in endoreplication, reflected in an increasing EI, see FIG. 10B. Whether this shift from cell division to endoreplication is successful to maintain homeostasis and the plants can cope with the stressful conditions or whether the plants suffer from the stress and the increasing EI displays a strong defence response to the Cd cannot be answered using the current parameters. Therefore, the different ploidy levels show a dose-dependent increase in the relative fraction of 8C whereas higher ploidy levels do not increase in comparison to the unexposed plants, FIG. 10C. This is confirmed by a dose-dependent increase in relative abundance ratios with respect to its subsequent ploidy level including ploidy levels of 8C and higher, FIG. 10D. Based on this, the tipping point ploidy level T for this plant system is set at 8C for Cd toxicity. Finally, the plants show a dose-dependent increase in EI_{DEFENCE}, see FIG. 10E, and a less pronounced dose-dependent decrease in EI_{GROWTH}, FIG. 10F. Hence, the increasing EI should be interpreted as plants that are in distress instead of in homeostasis. Here below, the calculations for the present example are proposed:

$${EI}_{DEFENCE} = {\sum }_{y = 0}^{y = a} ( {x}_{y} \times {Z}_{y} ) = {\sum }_{y = 0}^{y = 3} ( {x}_{y} \times {Z}_{y} ) = 0 \times {Z}_{0} + 0 \times {Z}_{1} + 1 \times {Z}_{2} + 2 \times {Z}_{3} \\ {EI}_{GROWTH} = {\sum }_{y = a + 1}^{y = k} ( {x}_{y} \times {Z}_{y} ) = {\sum }_{y = 4}^{y = 5} ( {x}_{y} \times {Z}_{y} ) = 3 \times {Z}_{4} + 4 \times {Z}_{5}$$

wherein A. thaliana is used, which system has a tipping point T equal to 8C, and therefore a=3, Z_{y}=fraction of P^{y}C, P=2 (diploid organism) and k=5 (highest ploidy level observed).

### Example 5: A. thaliana Leaves Cultivated in a Hydroponic System Exposed to Cadmium

To investigate whether the tipping point can also be determined using another cultivation system, A. thaliana plants were grown in a hydroponic system and exposed to a Cd concentration range after 11 days. Since results are promising for young seedlings, it was also investigated whether the invention works in plants of different ages. Therefore, individual samples of the first and the third leaf were harvested at different time points.

LEAF 1: FIG. 11 illustrates flow cytometry data of the first A. thaliana leaf exposed to a Cd range (1-10 μM Cd) after 11 days and harvested at different time points (12-21 days). Data are expressed relative to the unexposed condition (0 μM Cd=1.0). A) Endoreplication index (EI); B) Relative ploidy level; C) Relative ratios of consecutive ploidy levels; D) EI_{DEFENCE}; and E) EI_{GROWTH}. One-way ANOVA compared to the unexposed condition: * p<0.05. Exposure to Cd for 3 days (i.e., 14-days-old plants) or more results in a dose-dependent decrease of EI in leaf 1, see FIG. 11A. As known from literature, the maximum ploidy level of young leaves is lower than in mature plants. This is confirmed by the different ploidy levels observed, see FIG. 11B: leaf 1 reaches ploidy levels up to 8C after 12 days, 16C after 14 days and the highest ploidy level of 32C was only reached after 18 days. This shift in maximum ploidy level is hypothesized to influence the tipping point. Therefore, at each age, the tipping point is identified. After only one day of exposure to Cd (12-days-old plants), leaf 1 did not show significant changes in EI or ploidy levels, hence the tipping point is not relevant at this early stage. At day 14, a dose-dependent increase in the relative fraction of 4C was found whereas higher ploidy levels show a dose-dependent decrease, see FIG. 11B. This is confirmed by a dose-dependent increase in 4C/8C ratios, see FIG. 11C. Hence, the tipping point for leaf 1 after 14 days is set at 4C. At day 18 and 21, the relative fraction of 8C was strongly increased upon Cd exposure whereas higher ploidy levels decreased in comparison to the unexposed plants, see FIG. 11B. This is confirmed by increased 8C/16C ratios, see FIG. 11C. Hence, the tipping point for leaf 1 after 18 and 21 days is set at 8C, which is the same tipping point in seedlings grown in liquid MS medium that also achieve their maximal endoreplication capacity. Taking into account the different tipping points for the formulas, elevated values of EI_{DEFENCE}, see FIG. 11D, and decreased values of EI_{GROWTH}, see FIG. 11E, confirm the previous statements. Hence, the decreasing EI should be interpreted as plants that are in distress and not able to stimulate endoreplication to maintain homeostasis.

LEAF 3: FIG. 12 illustrates flow cytometry data of the third A. thaliana leaf exposed to a Cd range (1-10 μM Cd) after 11 days and harvested at different time points (14-21 days). Data are expressed relative to the unexposed condition (0 μM Cd=1.0). A) Endoreplication index (EI); B) Relative ploidy level; C) Relative ratios of consecutive ploidy levels; D) EI_{DEFENCE}; and E) EI_{GROWTH}. One-way ANOVA compared to the unexposed condition: * p<0.05. Leaf 3 was only present after 14 days but it shows analogous results as leaf 1. Leaves show a dose-dependent decrease in EI, see FIG. 12A. The different ploidy levels observed confirm the lower maximum ploidy level in young leaves, see FIG. 12B: leaf 3 reaches ploidy levels up to 8C after 14 days, 16C after 18 days and 32C after 21 days. At day 14, leaf 3 was focused on normal cell division as evidenced by the elevated 2C ratio. Hence, the tipping point is not relevant at this early stage. At day 18, leaf 3 shows increases in the relative fractions of 2C and 4C whereas higher ploidy levels are decreased, see FIG. 12B. This is confirmed by a dose-dependent increase in 4C/8C ratios, see FIG. 12C. Hence, the tipping point for leaf 3 after 18 days is set at 4C. At day 21, leaf 3 shows increases in the relative fractions of 2C, 4C and 8C whereas higher ploidy levels are decreased, see FIG. 12B. This is confirmed by a dose-dependent increase in the 8C/16C ratio, see FIG. 12C. Hence, the tipping point for leaf 3 after 21 days is set at 8C, which is the same tipping point in seedlings grown in liquid MS medium that also achieve their maximal endoreplication capacity. Taking into account the different tipping points for the formulas, elevated values of EI_{DEFENCE}, see FIG. 12D, and decreased values of EI_{GROWTH}, FIG. 12E, confirm the previous statements. Hence, the decreasing EI should be interpreted as plants that are in distress and not able to stimulate endoreplication to maintain homeostasis.

### Example 6: D. caryophyllus Seedlings Cultivated in MS Medium Exposed to Cadmium-Determination of Ploidy Level Tipping Point T

Since results are promising for young seedlings of A. thaliana, it was also investigated whether the invention works in other plant species. To investigate whether the tipping point can also be determined using another plant species, tetraploid D. caryophyllus plants (i.e., basic ploidy level is 4C) were grown in a 48-well microtiter plate with ¼ MS medium and exposed to a Cd concentration range between 0 and 50 μM Cd. FIG. 13 illustrates flow cytometry data of D. caryophyllus seedlings exposed to a Cd range (10-50 μM Cd) and harvested after 9 days. Data are expressed relative to the control condition shown by the horizontal line (1.0). A) Average number of nuclei per microliter of carrier fluid; B) Endoreplication index (EI); C) Relative ploidy level; D) Relative ratios of consecutive ploidy levels; E) EI_{DEFENCE}; and F) EI_{GROWTH}. One-way ANOVA compared to the unexposed condition: * p<0.05. Exposure to 50 μM Cd resulted in a decrease of nuclei concentration and in EI, see FIGS. 13A and 13B. Concerning the relative ploidy levels, a dose-dependent increase in the relative fraction of 4C was found whereas higher ploidy levels did not, see FIG. 13C. This is confirmed by a dose-dependent increase in 4C/8C ratios, see FIG. 13D. Hence, the tipping point for D. caryophyllus seedlings is set at 4C. Taking into account 4C as tipping point for the formulas, elevated values of EI_{DEFENCE}, see FIG. 11E, and decreased values of EI_{GROWTH}, see FIG. 11F, confirm the previous statements. Hence, the decreasing EI should be interpreted as plants that are in distress and not able to stimulate endoreplication to maintain homeostasis.

### Example 7: Additional Plant Types, Plant Parts, Growth-Promoting and Toxic Compounds Tested

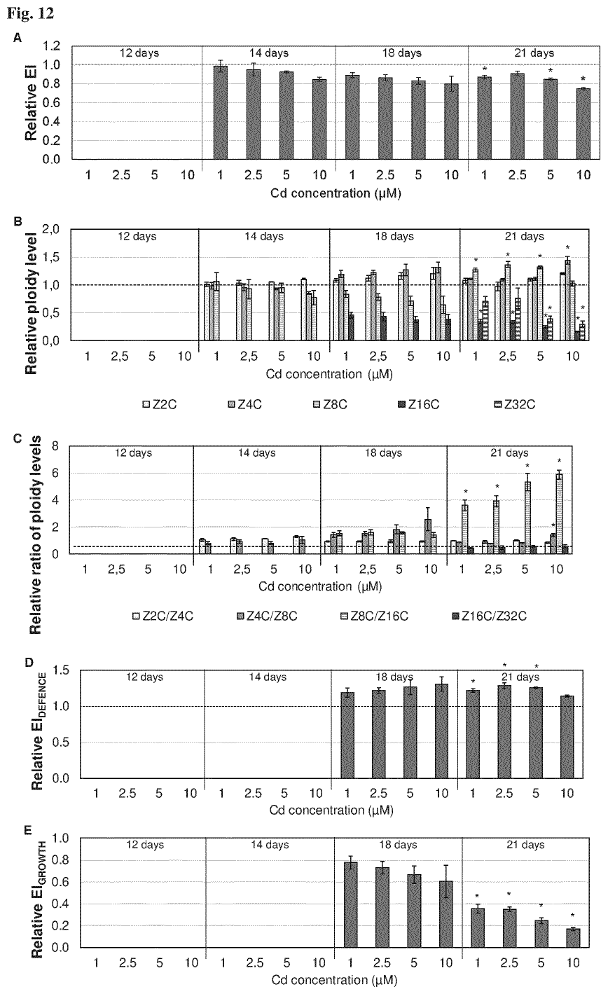

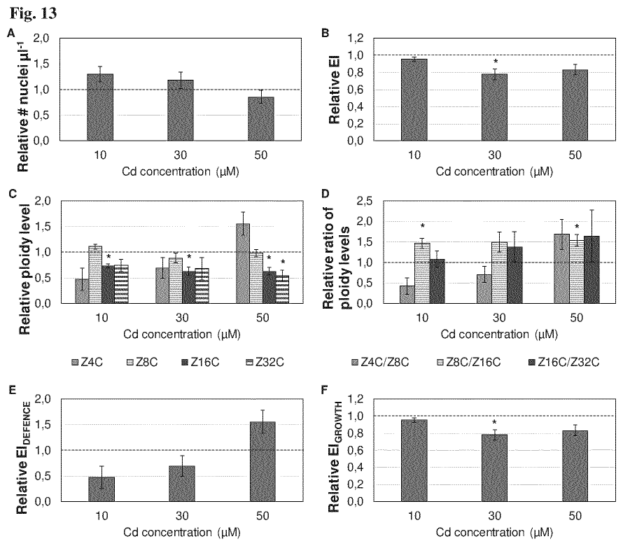

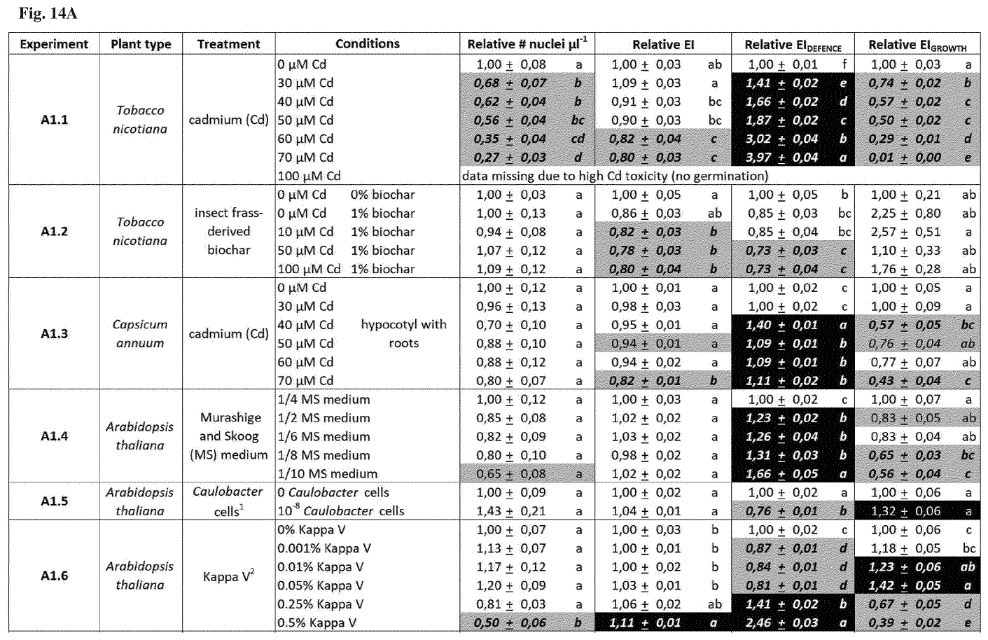

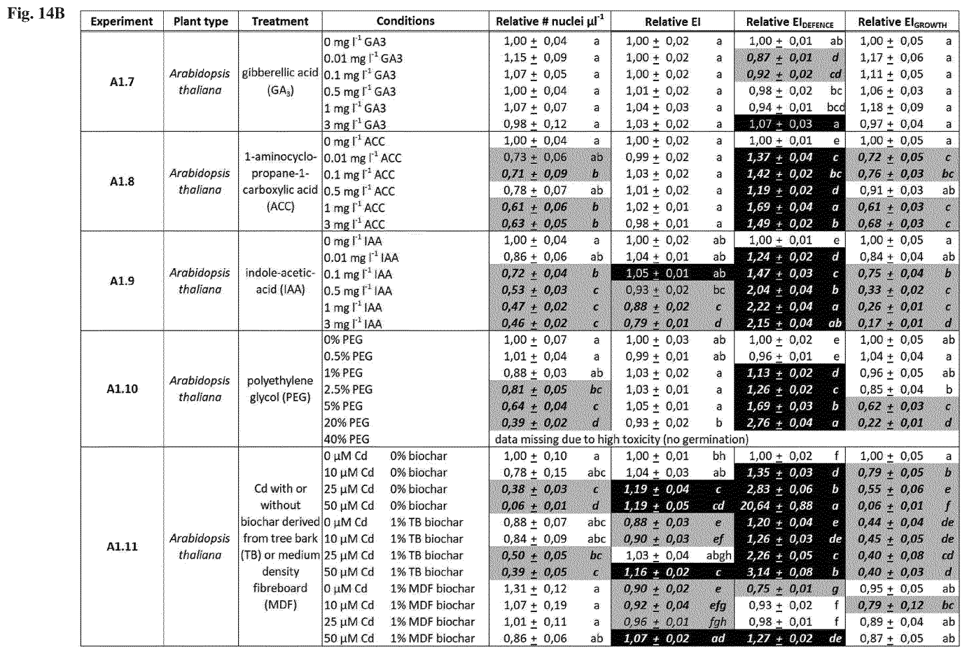

The present Example shows the method applicability of the present invention on two additional plant types (Tobacco nicotiana and Capsicum annuum), additional plant parts (hypocotyl with roots) and eight additional treatments. FIGS. 14A and B illustrates from left to right: Average number of nuclei per microliter of carrier fluid; Endoreplication index (EI), for defence (EI_{DEFENCE}) and for growth (EI_{GROWTH}) of the analysed plant samples from different plant systems exposed to the various treatments mentioned in the table. Data are expressed relative to the first listed condition of each experiment (reference). Different letters indicate significantly different values within each experiment (p<0.05), significant differences with respect to the first listed condition are highlighted in bold and italic (p<0.05), decreased values are coloured in (p<0.05 and p<0.1) and increased values are coloured in black (p<0.05 and p<0.1). In particular, the toxic effect of Cd in T. nicotiana (see Experiment A1.1) was confirmed by the present invention since EI_{GROWTH} decreased and EI_{DEFENCE} increased in the plant seedlings. Mitigation of this Cd-induced growth inhibition by biochar was confirmed (see Experiment A1.2). Also in C. annuum hypocotyls with roots, the toxic effect of Cd could be confirmed (see Experiment A1.3). To optimize the growing medium for A. thaliana, different dilutions of MS medium were tested (see Experiment A1.4). Based on the lowest EI_{DEFENCE}, accompanied by the highest EI_{GROWTH}, a dilution of ¼ was identified as the most optimal growing condition since both lower (½) and higher dilutions (⅙, ⅛, 1/10) showed a higher EI_{DEFENCE} and a lower EI_{GROWTH}. The growth-stimulating effects of the Caulobacter strain LMG P-31259 (see Experiment A1.5), Kappa V (trademark) (see Experiment A1.6) and GA3 (see Experiment A1.7) on A. thaliana were confirmed by the present invention since EI_{GROWTH} increased and/or EI_{DEFENCE} decreased. Interestingly, upon increasing concentrations of Kappa V and GA3, a shift towards toxicity was detected at 0.25% and 3 mg I-1, respectively, as indicated by both EI indices showing an opposite trend. The toxic effects of ACC (see Experiment A1.8), IAA (see Experiment A1.9) and the osmoticum PEG (simulating drought stress, see Experiment A1.10) on A. thaliana were confirmed by the present invention since EI_{GROWTH} decreased and EI_{DEFENCE} increased. Finally, Cd's toxic effect (see Experiment A1.11) and the prevention thereof by different biochar types was confirmed in A. thaliana by the present invention since EI_{GROWTH} decreased and EI_{DEFENCE} increased upon Cd exposure and the combination with both biochar types resulted in significant higher EI_{GROWTH} values and/or significant lower EI_{DEFENCE} values compared to the same Cd concentration without biochar.

### Conclusion

Based on the current data, we can confirm our previous hypotheses in the tetraploid D. caryophyllus concerning the different ploidy levels that act as a tipping point between growth/homeostasis and stress/defence. This difference can be statistically demonstrated in accordance with the method of the present invention, wherein 2 new endoreplication indices, EI_{GROWTH} and EI_{DEFENCE} are used. Further, the screening system (48-well) in accordance with the present invention is able to replace lengthy and labor-intensive experiments to predict the toxic or growth-promoting effect of biochar. Moreover, this prediction is independent of the growth matrix into which the compound or treatment is mixed. The method of the present invention can also be applied on other plant species, such as T. nicotiana and C. annuum.

### REFERENCES

1. Olszyk D M, Shiroyama T, Novak J M, Johnson M G. A Rapid-Test for Screening Biochar Effects on Seed Germination. Commun Soil Sci Plant Anal. 2018; 49(16):2025-2041. doi: 10.1080/00103624.2018.1495726. PMID: 30930464; PMCID: PMC6436638.
2. Poorter H, Fiorani F, Stitt M, Schurr U, Finck A, Gibon Y, Usadel B, Munns R, Atkin O K, Tardieu F, Pons T. The art of growing plants for experimental purposes: a practical guide for the plant biologist. Funct Plant Biol. 2012; 39:821-838. doi: 10.1071/FP12028.
3. Haeldermans T, Claesen J, Maggen J, Carleer R, Yperman J, Adriaensens P, Samyn P, Vandamme D, Cuypers A, Vanreppelen K, Schreurs S. Microwave assisted and conventional pyrolysis of MDF—Characterization of the produced biochars. J Anal Appl Pyrolysis. 2019; 138:218-230. doi: 10.1016/j.jaap.2018.12.027.
4. Hilber I, Blum F, Leifeld J, Schmidt H P, Bucheli T D. Quantitative determination of PAHs in biochar: a prerequisite to ensure its quality and safe application. J Agric Food Chem. 2012; 60(12):3042-3050. doi: 10.1021/jf205278v.
5. Hendrix S, Keunen E, Mertens A I G, Beemster G T S, Vangronsveld J, Cuypers A. Cell cycle regulation in different leaves of Arabidopsis thaliana plants grown under control and cadmium-exposed conditions. Environ Exp Bot. 2018; 155: 441-452. doi: 10.1016/j.envexpbot.2018.06.026.
6. Guijarro M, Pajares G, Riomoros I, Herrera P J, Burgos-Artizzu X P, Ribeiro A. Automatic segmentation of relevant textures in agricultural images. Comput Electron Agric. 2011; 75(1):75-83. doi: 10.1016/j.compag.2010.09.013.

1. A method for determining a toxicity and/or growth promotion effect of any one of a treatment or compound, comprising the steps of:
a) providing at least one plant system or parts thereof comprising at least one cell which ploidy is provided to change on exposure to the any one of a treatment or compound;
b) exposing the plant system or parts thereof to the treatment or compound;
c) providing at least a first and a second series of one or more ploidy levels of the nuclei in a sample of the exposed plant system or parts thereof, wherein
the first series is calculated from a basic ploidy level up to, and including, a ploidy level tipping point T corresponding to an arrest/inhibition towards higher ploidy levels for the sample; and
the second series is calculated from, and excluding, the ploidy level tipping point T to the highest ploidy level observed for the plant system; and
d) determining the toxicity and/or growth promotion effect of the treatment or compound in the plant system based on the first series and the second series of ploidy levels provided at step c).

2. The method according to claim 1, wherein the step d) of determining the toxicity and/or growth promotion effect is based on one or more of an endoreplication index (EI) calculated in accordance with formula (I) and/or formula (II)
$${EI}_{DEFENCE} = {\sum }_{y = 0}^{y = a} ( {x}_{y} \times {Z}_{y} ) \qquad ( I )$$
$${EI}_{GROWTH} = {\sum }_{y = a + 1}^{y = k} ( {x}_{y} \times {Z}_{y} ) \qquad ( II )$$
wherein EI_{DEFENCE} is a defence endoreplication index calculated in accordance with the first series provided at step c), and EI_{GROWTH} is a growth endoreplication index calculated in accordance with the second series provided at step c), and wherein
y is the index of each summation and a non-negative integer referring to the number of cell cycles, for which y≤1, or the number of endocycles plus 1, for which y>1,
x_{y} is an element of the real numbers,
Z_{y} is the fraction of nuclei in a sample of the exposed plant system or parts thereof of a represented ploidy level P^{y} DNA copies, wherein P is the basic ploidy level of said plant system,
k is equal to log_{P}(Q), wherein Q is the highest ploidy level of said plant system,
a is equal to log_{P}(T), wherein T is the ploidy level tipping point of said plant system.

3. The method according to any one of the previous claims, wherein the ploidy level tipping point T is defined as the lowest ploidy level for which its relative abundance ratio with respect to its subsequent ploidy level, shows a dose-dependent increase with the treatment or compound within a certain range.

4. The method according to any one of the previous claims, wherein at step d) the toxicity effect is determined based on the first series provided at step c) or EI_{DEFENCE}, and/or the growth promotion effect is determined based on the second series provided at step c) or EI_{GROWTH}.

5. The method according to claim 4, wherein the toxicity effect on the plant system is determined by means of EI_{DEFENCE} of the plant system being higher than a EI_{DEFENCE} for a control, and/or the growth promotion effect on the plant system is determined by means of EI_{GROWTH} for the plant system being higher than a EI_{GROWTH} for a control.

6. The method according to any one of the previous claims, wherein at step a) the plant system is provided in a growth matrix to which the treatment or compound of step b) is applied.

7. The method according to claim 6, wherein the growth matrix is a liquid medium, soil or substrate of the plant system.

8. The method according to any one of the previous claims, wherein at step a) the plant system is selected from: Arabidopsis thaliana, Dianthus caryophyllus, preferably Arabidopsis thaliana.

9. The method according to any one of the previous claims, wherein at step b) the sample of the exposed plant system is a seedling of the plant system.

10. The method according to any one of the previous claims, wherein at step d) the toxicity and/or growth promotion effect of the treatment or compound is determined based on data measured by means of flow cytometry.

11. The method according to any one of the previous claims, wherein at step a) the plant system is provided in a microtiter plate.

12. The method according to any one of the previous claims, wherein at step b) the compound is a carbonized material, such as conventional pyrolysis (CPS) carbonized material, conventional pyrolysis including a washing step and degassing after grinding (CPS-W/OG) obtained carbonized material or microwave pyrolysis (MWP) obtained carbonized material.

13. A computer program comprising instructions which, when the program is executed by a computer, realises the computer to carry out the steps c) and d) of the method of claims 1-5.
